(19) United States
(12) Patent Application Publication
(10) Pub. No.: US 2013/0060074 A1
(43) Pub. Date: Mar. 7, 2013

(54) Method for Producing High Purity Biomethane Without Adding Unacceptable Quantities of Moisture

(71) Applicant: Stearns Conrad Schmidt Consulting Engineers, In, Long Beach, CA (US)

(72) Inventor: Jeffrey L. Pierce, Torrance, CA (US)

(73) Assignee: STEARNS CONRAD SCHMIDT CONSULTING ENGINEERS, INC., Long Beach, CA (US)

(21) Appl. No.: 13/668,476

(22) Filed: Nov. 5, 2012

(51) Int. Cl. C07C 7/12

(52) U.S. Cl. 585/802

(57) ABSTRACT

A method for producing high purity biomethane using two non-regenerative media in series to reliably and virtually completely eliminate undesirable impurities from biomethane. The first media in the series is activated carbon, preferably virgin media, produced from coconut shells or coal. The second media in the series is silica gel or other desiccant. Neither material is regenerated; rather, the purity of the biomethane is monitored and when the media is spent, the media is discarded.

### BACKGROUND OF THE INVENTION

Biogas (methane-containing gas produced by biological processes, such as landfill gas, municipal wastewater treatment plant digester gas and other digester gases) is increasingly being seen as a valuable source of renewable energy. After suitable processing, biogas can be used as a natural gas substitute, as a compressed natural gas substitute, and to generate electric power in conventional power plants. Once processed into a form that can be used in such applications, the converted biogas is known as biomethane. Biogas conversion technologies currently in use include membrane technology, pressure swing adsorption technology, absorption technology (e.g., Selexol) and water wash, among others.

This invention relates to the purification of biomethane. The invention reduces concentrations of siloxanes, volatile organic compounds, metals and other impurities to non-detectable or nearly non-detectable concentrations. The purification allows use of the biomethane for purposes that it would otherwise be excluded from use. While accomplishing this purification, a component of the invention prevents the addition of unacceptable quantities of moisture, which would otherwise render the biomethane unusable for the uses that the purification was intended to achieve.

Technologies currently in use to convert biogas into biomethane focus on removal of carbon dioxide, nitrogen and oxygen. Consistent removal of other undesirable impurities cannot be expected, or guaranteed, particularly as the equipment and media used in these technologies approaches their scheduled maintenance intervals.

Increasingly, biomethane applications require extremely high purity biomethane. One example is production of a pipeline quality gas equivalent. Several conventional technologies are in use to convert biogas to pipeline quality gas equivalent, including pressure swing adsorption, membrane separation technology and liquid absorption technology. Some pipeline companies are now requiring close to non-detectable concentrations of impurities (i.e., siloxanes, other volatile organic compounds, metals and other impurities). These impurities still exist in biomethane, at the part per million or part per billion level after conventional biogas processing. The impurity concentrations now being required are often below the levels that conventional technologies for processing biogas into biomethane can consistently and reliably achieve. A second example is the use of biomethane in a power generation application where the power generation equipment, or its air pollution control equipment, is very sensitive to these impurities, and the power generator has required the equivalent of pipeline quality gas, even if the biomethane is being used on-site, and does not actually enter a pipeline company pipeline. A third example is the use of biomethane as a vehicle fuel in the form of compressed natural gas. Some of these biomethane uses (e.g., use as pipeline quality gas or use as a vehicle fuel) always require that a very low level of moisture be maintained in the product biomethane. It is, therefore, of the utmost importance that the advanced biomethane purification process not add unacceptable quantities of moisture to the biomethane. The above three examples are not intended to cover all applications that the invention could apply to, but are identified as examples of where the invention could immediately be usefully applied in the biomethane industry.

The present invention accomplishes the goals of advanced purification of the biomethane to meet very high impurity removal standards, as an add-on step to conventional biomethane processing technologies, but while achieving advanced purification, it does not add unacceptable quantities of moisture to the biomethane.

The invention employs non-regenerative (i.e., single-use) activated carbon and a non-regenerative desiccant in series, as an add-on process, to any conventional biomethane processing technology. Activated carbon contains moisture, as a result of its manufacturing process. Without being coupled with the desiccant step, provided in this invention, the biomethane would be free of impurities, but would be unsuitable for use, because it would exceed the moisture limitations governing the biomethane's end use.

### SUMMARY OF THE INVENTION

An object of the invention is to provide a simple method for purifying biomethane, while controlling its water content.

This object is attained by a method for producing high purity biomethane as described below.

The present invention is a two-stage process.

The first-stage consists of vessels operating in parallel or in series. The vessels contain a non-regenerative adsorbent media. Once the media is exhausted, as detected by the beginning of the rise in impurities in the biomethane exiting the vessels (i.e., breakthrough), the media is replaced by fresh media. The quantity and type of media are selected based on biomethane flow rate, inlet impurity concentrations and desired outlet impurity concentrations (i.e., end user specification).

The media required in the first-stage is preferably activated carbon (coconut shell or coal based, of a selected CTC number), sometimes supplemented with a blend of other adsorbents. The first-stage removes the undesirable impurities. Breakthrough is detected by monitoring the product gas with periodic measurement for volatile organic compounds using colorimetric tubes, or by online analyzer, or by laboratory tests.

The second-stage of the invention consists of a vessel or vessels operating in parallel. These vessels contain a non-regenerative media (e.g., silica gel) that selectively adsorbs water. The media quantity is selected based on the expected residual moisture from the stage-one vessels and the flow rate and the moisture standard imposed by the end user. For example, the standard pipeline company limitation for water is 7 lbs/MMscf. “MMscf” means “million standard cubic feet”.

### BRIEF DESCRIPTION OF THE DRAWING

In the accompanying drawing,

FIG. 1 is a process flow schematic of a method for producing high purity biomethane without adding unacceptable quantities of moisture embodying the invention and

FIG. 2 is an alternative embodiment of the process.

### DESCRIPTION OF THE PREFERRED EMBODIMENT

A method for producing high purity biomethane without adding unacceptable quantities of moisture embodying the invention is illustrated schematically in the drawing.

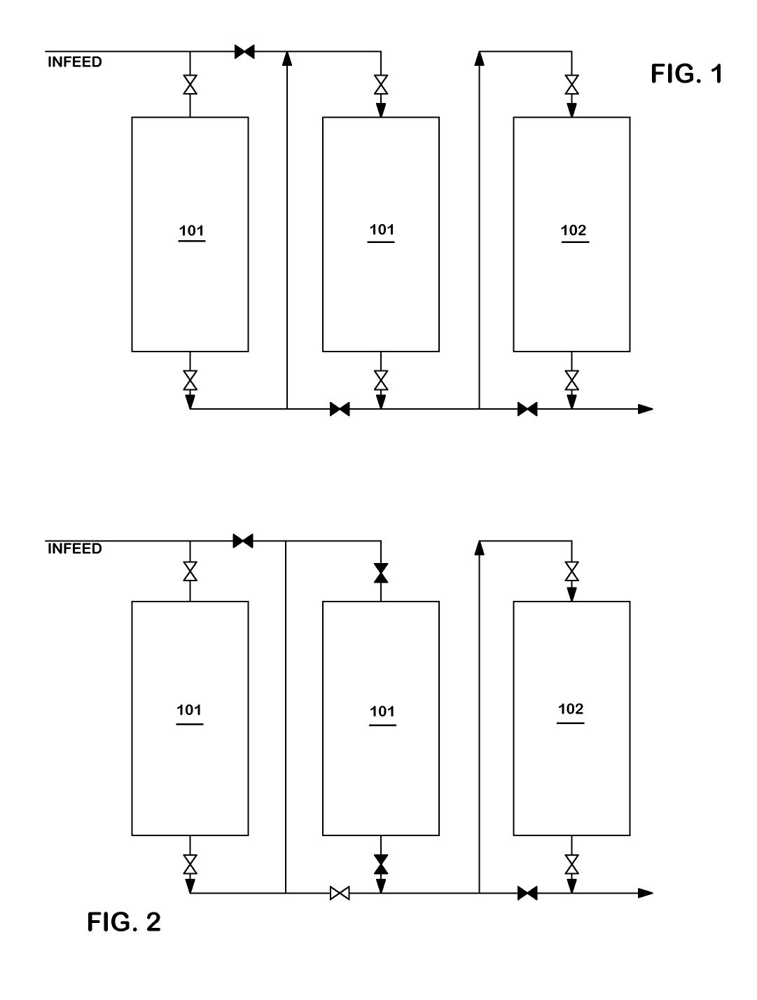

In FIGS. 1 and 2, reference 101 designates biomethane purification vessels. Two such purification vessels are shown side-by-side; however, a greater number of purification vessels 101, or only one, might be used. When multiple purification vessels are used, they can be placed in parallel or in series.

If the vessels 101 are in parallel, as shown in FIG. 2, they can be piped to allow only one vessel to be online at a time. Note: valves at the top and bottom of each vessel are shown schematically; the valves shown in solid black are closed, the others are open. Alternatively, the valves at the top and bottom of the center vessel could be open, in which case both vessels 101 would be online together.

If the vessels 101 are in series, as shown in FIG. 1, they can be piped to allow only sequential forward flow (as shown), or to allow the vessels to be reversed. Reversal would allow the second vessel to become the first, and require that only the “new” second vessel be filled with fresh media, after impurity breakthrough.

The purification vessel(s) are filled with an adsorbent media that removes volatile organic compounds and other impurities. The adsorbent media is preferably conventional activated carbon (coconut shell or coal based, of a selected CTC number), possibly supplemented with a blend of other adsorbents. The first stage removes the undesirable impurities. Breakthrough is detected by monitoring the product gas with periodic measurement for volatile organic compounds using colorimetric tubes or an online analyzer, or by laboratory tests.

Reference 102 designates biomethane dehydration vessels. Each of the figures shows only one dehydration vessel; however, multiple vessels may be used. If multiple dehydration vessels are used, they would be placed in parallel. In parallel, they can be piped to allow only one vessel or both vessels to be online at a time.

The dehydration vessel is filled with a desiccant, such as silica gel. The water content of the biomethane exiting the dehydration vessels is monitored. When the water content approaches the acceptable limit (i.e., the desiccant is nearly saturated with water), it is discarded and replaced by fresh desiccant.

The drawing and the description above are not intended to limit the scope of the invention. The invention can be implemented with any number of tanks, and with other piping arrangements, in order to adapt the design to a specific application. The basic concept of the present invention is using two non-regenerative media in series to reliably—and virtually completely—eliminate undesirable impurities from biomethane, without creating a moisture problem in the biomethane, when removing the impurities.

Since the invention is subject to modifications and variations, it is intended that the foregoing description and the accompanying drawings shall be interpreted as only illustrative of the invention defined by the following claims.

What is claimed is:

1. A method for purifying biomethane, said method comprising steps of removing siloxanes, volatile organic compounds, metals and other low concentration impurities by passing the biomethane through a purification vessel containing a material which adsorbs said impurities, and then
removing water from said biomethane by passing the biomethane through a dehydration vessel containing a desiccant which absorbs water.

2. The method of claim 1, wherein the activated carbon is produced from coconut shells or coal.

3. The method of claim 2, wherein the activated carbon contains substantial moisture.

4. The method of claim 1, further comprising steps of monitoring the material which absorbs impurities, and replacing the material when breakthrough is detected.

5. The method of claim 4, is monitoring using colorimetric tubes, or online analyzer, or laboratory tests.

6. The method of claim 1, wherein the desiccant is silica gel, or similar desiccant.

7. The method of claim 4, further comprising steps of monitoring the material which absorbs the water, and replacing it when water breakthrough is approaching undesirable levels.

8. The method of claim 7, is monitoring using colorimetric tubes, or online analyzer, or laboratory tests.
